(19) United States
(12) Patent Application Publication
(10) Pub. No.: US 2011/0015658 A1
(43) Pub. Date: Jan. 20, 2011

(54) Eye Surgery Instrument

(75) Inventor: Gerrit Jan Vijfvinkel, Geervliet (NL)

Correspondence Address: SWANSON & BRATSCHUN, L.L.C., 8210 SOUTHPARK TERRACE, LITTLETON, CO, 80120, US

(73) Assignee: D.O.R.C. Dutch Ophthalmic Research Center (International) B.V., Zuidland (NL)

(21) Appl. No.: 12/811,677

(22) PCT Filed: Mar. 25, 2009
(86) PCT No.: PCT/NL2009/050142

§ 371 Date: Oct. 8, 2010

Related U.S. Application Data
(60) Provisional application No. 61/039,458, filed on Mar. 26, 2008.

(30) Foreign Application Priority Data
Mar. 25, 2008 (NL) 2001401

(51) Int. Cl. A61F 9/007

(52) U.S. Cl. 606/166

(57) ABSTRACT

The invention relates to an eye surgery instrument, comprising an entry module provided with a sharp distal end. Furthermore, the instrument comprises an operating element for manipulating the eye surgery instrument. The operating element is attached to the entry module via a curved segment. The curved segment preferably comprises a kink.

The invention relates to an eye surgery instrument, comprising an entry module provided with a sharp distal end and an operating element for manipulating the instrument.

Such an eye surgery instrument is for instance known from United States patent publication US 2004/0073231. During an eye surgical procedure, the entry module of the instrument is inserted with the aid of the operating element via an incision in the conjunctiva and the sclera into the intra-ocular space of the eye. Thereupon, the entry module can receive surgical aids which extend into the intra-ocular space to enable surgical procedures to be performed. Since an incision of relatively limited dimensions is provided, the tissue can rapidly recover following the intervention, without the use of sutures.

However, manipulating such an eye surgery instrument requires quite some skill and is sometimes hampered by projecting parts near the patient's eye, such as the bridge of the nose.

The invention contemplates an eye surgery instrument of the type mentioned in the opening paragraph, which, while preserving the advantages, obviates the drawback mentioned. In particular, the invention contemplates obtaining an eye surgery instrument which features enhanced manipulability. To this end, the operating element is attached to the entry module via a curved segment.

By attaching the operating element to the entry module via a curved segment, the surgeon can avoid contact with parts of the face near the eye by pivoting the operating element. Thus, the freedom of movement of the instrument increases and manipulability is enhanced.

Preferably, the curved segment comprises a kink, so that the curvature extends over a relatively short length. This involves a relatively small radius of curvature. Consequently, the effect of the curvature, viz. affording a greater freedom of movement, is utilized optimally. It is, of course, also possible to make the curved segment of different design, for instance as a bent segment with a relatively large radius of curvature. In addition, the curved segment can comprise a plurality of non-straight parts, for instance with different radii of curvature and/or orientations.

Further advantageous embodiments of the invention are set forth in the subclaims.

The invention will be further elucidated with reference to exemplary embodiments which are represented in the drawing. In the drawing:

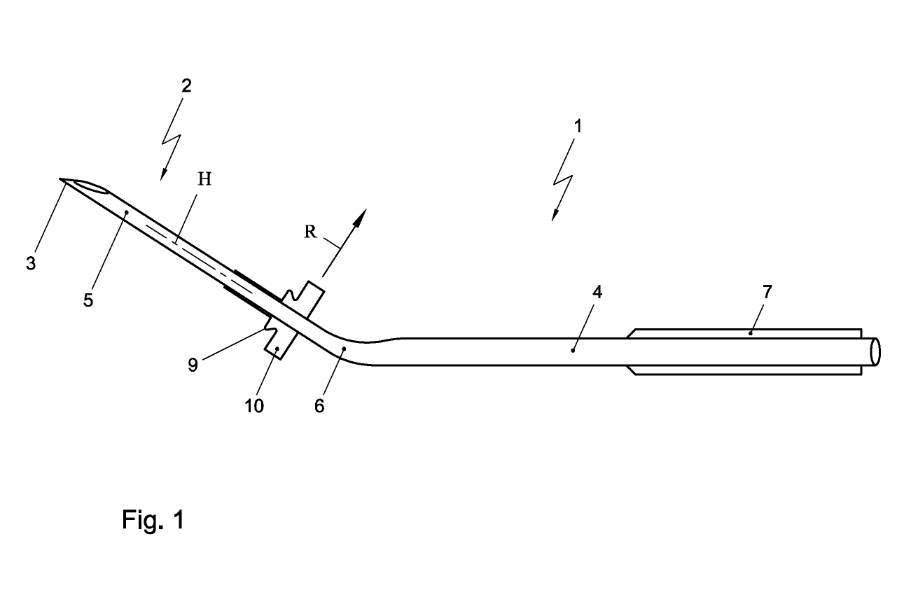

FIG. 1 is a schematic elevational view of a cross section of a surgical instrument according to the invention; and

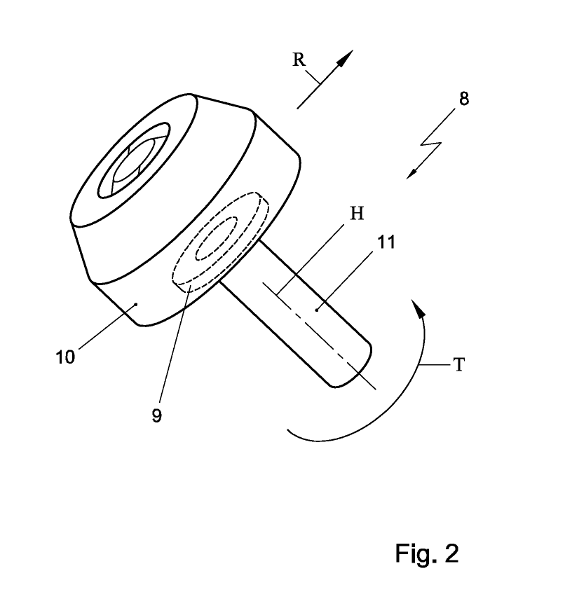

FIG. 2 is a schematic perspective view of a sealing module of the instrument of FIG. 1.

The figures are only schematic representations of a preferred embodiment of the invention. In the figures, the same or corresponding parts are indicated with the same reference numerals.

FIG. 1 shows a schematic view of a cross section of a surgical instrument 1 according to the invention. The instrument 1 comprises an entry module 2 provided with a sharp distal end 3 and an operating element 4 for manipulating the instrument 1. The entry module 2 comprises a substantially tubular element 5 which is of substantially straight design. The operating element 4 is also substantially straight. It is noted that instead of a tubular element 5 also a solid pin-shaped element may be used.

The operating element 4 and the entry module 2 are attached to each other via a curved segment 6 which comprises a kink. Furthermore, the curved segment is integrally formed with the operating element 4 and the entry module. 2. The operating element 4, the curved segment and the entry module 2 hence form one whole. In other embodiments according to the invention, the curved segment is formed integrally only with the operating element 4 or the entry module 2, or is even of wholly discrete design. Furthermore, the operating element 4 of the instrument 1 is provided with a grip 7.

In a specific embodiment, the operating element 4 is detachably attached to the entry module 2.

According to an aspect of the invention, the entry module 2 is provided with a sealing module 8 which surrounds the tubular element 5 in circumferential direction. FIG. 2 shows a schematic perspective view of such a sealing module 8.

The sealing module 8 comprises a substantially stiff flange 9 which extends from the tubular element 5 in radial direction R, substantially transversely to the centerline H of the tubular element 5. Furthermore, the sealing module 8 comprises an elastic collar 10 which surrounds the tubular element 5 in circumferential direction T. The stiff flange 9 contributes to the dimensional stability of the sealing module 8.

Preferably, the sealing module 8 furthermore comprises a carrying element 11 slidable in axial direction relative to the centerline H of the tubular element 5, to which the flange 9 and the elastic collar 10 are attached. In this way, the axial position of the sealing module 8 with respect to the end 3 of the entry module 2 can be set as desired.

During use of the eye surgery instrument 1 the surgeon makes incisions in the conjunctiva and the sclera with the sharp end 3 of the entry module 2, the entry module 2 being introduced into the intra-ocular space, so far that the sealing module described sealingly abuts against the eye tissue around the incisions. The carrying element 11 of the sealing module 8 then also extends into the intra-ocular space. Next, the entry module 2 is removed from the eye, leaving the sealing module behind.

Since the sealing module 8 remains behind in the eye tissue, the incisions in the conjunctiva and the sclera remain aligned with respect to each other, so that undue stress in the tissue is prevented. Furthermore, the sealing module can receive a surgical module which extends into the intra-ocular space. Thus, the eye surgeon can perform surgical procedures in the intra-ocular space in a non-invasive manner. The surgical module may for instance comprise a cutting module, a laser module or a lighting module. Also, the sealing module may be utilized for performing visual inspections, or for introducing or extracting liquid and/or gas into and out of the intra-ocular space.

The elastic properties of the collar 10 provide for a sealing action against the eye tissue, in order to prevent ocular fluid leaking out from the intra-ocular space of the eye through the incisions. In addition, the stiff flange 9 prevents an unwanted contraction and/or dilation of tissue around the incision.

The interior of the sealing module is dimensioned such that surgical modules with a specific outside dimension, for instance 20, 23 or 25 Gauge can be received by the sealing module.

Following completion of the surgical procedures in the intra-ocular space of the eye, also the sealing module 8 is removed. Owing to the slight dimensions of the incision, the wound is self-sealing.

It is noted that the tubular element 5 and the flange 9 are preferably manufactured from stainless steel, yielding a product that is easy to sterilize. Also, the tubular element and the flange may be manufactured from other materials, for instance from titanium and/or plastic.

The invention is not limited to the exemplary embodiments described here. Many variants are possible.

For instance, the curved segment of the instrument may be of rigid or somewhat resilient design.

Furthermore, the instrument may comprise a blunt distal end instead of a sharp distal end.

Such variants will be clear to those skilled in the art and are understood to be within the scope of the invention as set forth in the following claims.

1. An eye surgery instrument, comprising an entry module provided with a sharp distal end and an operating element for manipulating the instrument, wherein the operating element is attached to the entry module via a curved segment.

2. The eye surgery instrument according to claim 1, wherein the curved segment comprises a kink.

3. The eye surgery instrument according to claim 1, wherein the curved segment is integrally formed with at least one of the operating element and the entry module.

4. The eye surgery instrument according to claim 1, wherein the entry module comprises one of a substantially tubular and a pin shaped element.

5. The eye surgery instrument according to claim 4, wherein the entry module is provided with a sealing module which surrounds the one of a tubular element and a pin shaped element in a circumferential direction.

6. The eye surgery instrument according to claim 5, wherein the sealing module comprises a substantially stiff flange which extends from the one of a tubular element and a pill shaped element in a radial direction.

7. The eye surgery instrument according to claim 5, wherein the sealing module furthermore comprises an elastic collar which surrounds the one of a tubular element and a pin shaped element in a circumferential direction.

8. The eye surgery instrument according to claim 9, wherein the sealing module furthermore comprises a carrying element slidable in axial direction relative to a centerline of the one of a tubular element and a pin shaped element, to which the flange and the elastic collar are attached.

9. The eye surgery instrument according to claim 6, wherein the sealing module furthermore comprises an elastic collar which surrounds the one of a tubular element and a pin shaped element in a circumferential direction.
